(19) United States
(12) Patent Application Publication
(10) Pub. No.: US 2022/0378568 A1
(43) Pub. Date: Dec. 1, 2022

(54) KINK-RESISTANT TUBULAR SCAFFOLDS WITH ENHANCED RADIAL STRENGTH FOR TISSUE ENGINEERING APPLICATIONS

(71) Applicant: Matregenix, Inc., Irvine, CA (US)

(72) Inventor: Sherif Soliman, Irvine, CA (US)

(73) Assignee: Matregenix, Inc., Irvine, CA (US)

(21) Appl. No.: 17/827,643

(22) Filed: May 27, 2022

Related U.S. Application Data
(60) Provisional application No. 63/193,830, filed on May 27, 2021.

(51) Int. Cl. A61F 2/04

(52) U.S. Cl. CPC A61F 2/04; A61F 2002/046; A61F 2002/043; A61F 2002/044; A61F 2/07

(57) ABSTRACT

A tubular construct that includes a braided tube embedded therein is disclosed herein. The braided tube may be embedded between layers of the tubular construct or may alternatively be positioned flush with the inside of the tubular construct. The tubular construct is resistant to kinking and has enhanced radial strength. The braided tube reinforces the wall of the tubular construct by improving burst pressure resistance, tube strength, and torque transmission. When radial pressure is applied to the braided tube that is embedded in the construct, the braided tube cannot expand lengthwise. Thus, the compression strength of the construct is increased in the radial direction. This feature takes advantage of the same principle used in the children's toy colloquially known as a Chinese finger trap. The increased radial strength of the tubular construct prevents the construct from collapsing and thereby enhances its structural integrity.

### CROSS-REFERENCE TO RELATED APPLICATIONS

This application claims the benefit of U.S. Provisional Patent Application Ser. No. 63/193,830, filed on May 27, 2021, the disclosure of which is hereby incorporated in its entirety herein by reference.

### BACKGROUND

### Field of the Invention

The present disclosure relates to tubular constructs for tissue engineering applications.

### Description of the Related Art

Synthetic tubular constructs are used in various tissue engineering applications, including tracheal, bronchial, and esophageal scaffolds and vascular and urinary tract grafts and scaffolds. A commonly encountered problem is that of kinking under mechanical stress. Kinking jeopardizes the physical and mechanical integrity of the construct, leading to potential failure or collapse.

Thus there is a need for a tubular construct for use in tissue engineering applications that is resistant to kinking when mechanical stress is applied.

### SUMMARY

A tubular construct that includes a braided tube embedded therein is disclosed herein. The braided tube may be embedded between layers of the tubular construct or may alternatively be positioned flush with the inside of the tubular construct. The tubular construct is resistant to kinking and has enhanced radial strength. The braided tube reinforces the wall of the tubular construct by improving burst pressure resistance, tube strength, and torque transmission. When radial pressure is applied to the braided tube that is embedded in the construct, the braided tube cannot expand lengthwise. Thus, the compression strength of the construct is increased in the radial direction. This feature takes advantage of the same principle used in the children's toy colloquially known as a Chinese finger trap. The increased radial strength of the tubular construct prevents the construct from collapsing and thereby enhances its structural integrity.

### BRIEF DESCRIPTION OF THE DRAWINGS

The figures provided herewith are intended to illustrate but not to limit the invention.

FIG. 1 shows an embodiment of the tubular construct with the braided tube embedded between layers of the construct.

FIG. 2 shows representative cross-sectional views of a coated braided tube and an uncoated braided tube.

FIG. 3 shows an embodiment of a continuous coiled tube.

### DETAILED DESCRIPTION

A tubular construct that includes a braided tube embedded therein is disclosed herein. The braided tube may be embedded between layers of the tubular construct or may alternatively be positioned flush with the inside of the tubular construct. The tubular construct is resistant to kinking and has enhanced radial strength. The braided tube reinforces the wall of the tubular construct by improving burst pressure resistance, tube strength, and torque transmission. When radial pressure is applied to the braided tube that is embedded in the construct, the braided tube cannot expand lengthwise. Thus, the compression strength of the construct is increased in the radial direction. This feature takes advantage of the same principle used in the children's toy colloquially known as a Chinese finger trap. The increased radial strength of the tubular construct prevents the construct from collapsing and thereby enhances its structural integrity.

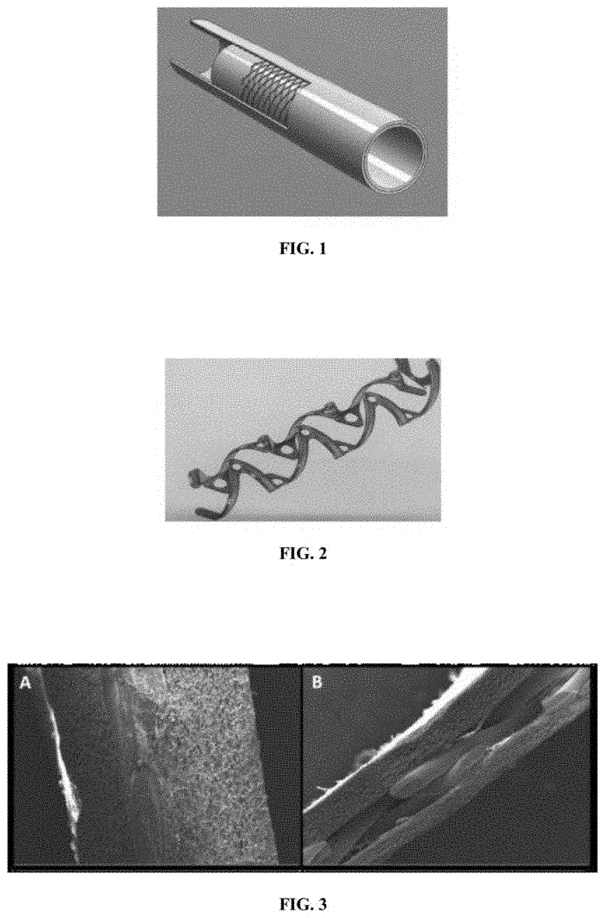

In some embodiments, the braided tube is embedded between layers of the tubular construct. In such embodiments, the layers of the tubular construct act as permissive substrates for the anchoring of the braided tube within the construct. FIG. 1 shows an embodiment of the tubular construct with the braided tube embedded between layers of the construct.

The braided tube may be fabricated with a wire diameter, braiding angle, and mesh that is appropriate to generate a tubular construct of desired flexibility and radial strength. This allows adjustment of the mechanical properties of the tubular construct to be suitable for the specific application in which it will be used. Typically, use of a smaller braiding angle will result in a braided tube that is stiffer and has higher torque transmission and reduced stretchability, whereas use of a larger braiding angle will result in a braided tube with increased flexibility and kink resistance.

In some embodiments, the braided tube is fabricated with monofilament braids. In other embodiments, the braided tube is fabricated with multifilament braids.

In some embodiments, the braided tube is fabricated to be substantially circular. In other embodiments, the braided tube is fabricated to be substantially D-shaped.

The braided tube may be formed from metal or a polymer.

In some embodiments, the braided tube is a continuous coiled tube composed of metal or polymer that does not have distinctly separated braids. FIG. 2 shows an embodiment of a continuous coiled tube that may be used as a braided tube of the tubular construct.

In some embodiments, the tubular construct is composed of a braided tube and a polymer having fibrous layers that is generated using electrospinning. Electrospinning allows for adjustment of the morphological characteristics of the polymer such as the thickness, packing density, orientation, and microstructure characteristics of the fibrous layers, thereby facilitating tuning of the mechanical properties of the tubular construct, including its tensile strength, compression strength, ability to elongate, range of motion, torsional strength, resistance to bending and rotation, compliance, degrees of freedom, and other mechanical properties.

In some embodiments, the mechanical integrity and binding forces between the polymer and the braided tube and between layers of the polymer are enhanced by electrospraying short fibers prior to electrospinning the subsequent layer. In some embodiments, the mechanical integrity and binding forces between the polymer and the braided tube and between layers of the polymer are enhanced by using a low viscosity polymer solution for electrospinning. In some other embodiments, the mechanical integrity and binding forces between the polymer and the braided tube and between layers of the polymer are enhanced by electrospinning wet fibers by decreasing the screen distance to generate a “tacky surface” prior to electrospinning the subsequent layer.

In some embodiments, the braided tube is coated. A coating on the braided tube may serve as a substrate for electrospinning subsequent layers onto the braided tube. A coating will also decrease the likelihood of voids forming between the polymer and the braided tube.

In other embodiments, the braided tube is uncoated.

FIG. 3 shows representative cross-sectional views of a coated braided tube (A) and an uncoated braided tube (B).

During fabrication of the tubular construct, the braided tube may be held under tension from the ends of the braided tube while subsequent polymer layers are applied, resulting in elongation of the braided tube during fabrication. Release of this tension at the end of the fabrication process will affect the amount of polymer in the tubular construct and thereby affect its mechanical properties. In some embodiments, tension is applied to the braided tube only during portions of the fabrication process. In some embodiments, the application of tension is computer-controlled and based on data collected during the fabrication process.

In some embodiments, the braided tube has properties that enhance its binding to the fiber layers which it contacts.

In a typical electrospinning apparatus, electrospun fibers are collected on a rotating metal drum of either cylindrical or semicircular shape, known as a mandrel. Mandrels are typically composed of two pieces, a long, thin, conductive metal rod that connects at its end to a rotating motor of the electrospinning apparatus and a nonconductive polymer cover that covers the metal rod.

In some embodiments, an applicator is secured to the mandrel prior to electrospinning. The applicator facilitates removal of the tubular construct from the mandrel. The applicator is preferably a braided tube. Tension may be applied along the main axis of the applicator to ensure a secure attachment to the mandrel. The applicator is then preferably secured to the mandrel using fasteners. The fasteners may, for example, be cable ties. Polymer layers are then electrospun onto the applicator. The braided tube of the tubular construct is then secured to the electrospun layers, and additional polymer layers are then electrospun over the braided tube to form a tubular construct. The tubular construct is composed of polymer layers with a braided tube embedded therein. The fasteners are removed to facilitate removal of the applicator from the mandrel. Tension is then applied to both ends of the applicator to facilitate its removal from the inside of the tubular construct. Upon removal of the applicator from the tubular construct, the fully fabricated tubular construct is ready for use in the desired application or further processing such as sizing.

In some embodiments, the tubular scaffold is resorbable in vivo, where both the polymer and the braided tube are resorbable. In some other embodiments, the polymer within the tubular scaffold is resorbable in vivo, but the braided tube is non-resorbable in vivo. In yet other embodiments, the tubular scaffold is non-resorbable in vivo.

In some embodiments, the tubular scaffolds are fabricated using a method selected from the group consisting of electrospinning, 3D printing, solvent casting, gas foaming, phase separation, fiber bonding, and freeze drying.

The previous description of the disclosed embodiments is provided to enable any person skilled in the art to make or use the invention disclosed herein. Although the various inventive aspects are disclosed in the context of one or more illustrated embodiments, implementations, and examples, it should be understood by those skilled in the art that the invention extends beyond the specifically disclosed embodiments to other alternative embodiments and/or uses of the invention and obvious modifications and equivalents thereof. It should be also understood that the scope of this disclosure includes the various combinations or sub-combinations of the specific features and aspects of the embodiments disclosed herein, such that the various features, modes of implementation, and aspects of the disclosed subject matter may be combined with or substituted for one another. The generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

All references cited are hereby expressly incorporated herein by reference.

What is claimed is:

1. A tubular construct comprising a braided tube enclosed within a polymeric outer tube, wherein the polymeric outer tube is formed by electrospinning.
